(19) United States
(12) Patent Application Publication
(10) Pub. No.: US 2022/0259240 A1
(43) Pub. Date: Aug. 18, 2022

(54) PROCESS FOR THE PREPARATION OF RUTHENIUM COMPLEXES

(71) Applicant: JOHNSON MATTHEY PUBLIC LIMITED COMPANY, London (GB)

(72) Inventors: Daniel J ARNOLD, West Deptford, NJ (US); Thomas COLACOT, West Deptford, NJ (US); Ben GARDNER, Royston (GB); Carin SEECHURN, Cambridge (GB); Peter D TANCINI, West Deptford, NJ (US)

(21) Appl. No.: 17/594,899

(22) PCT Filed: Aug. 13, 2020
(86) PCT No.: PCT/GB2020/051935

§ 371 Date: Nov. 2, 2021

Related U.S. Application Data
(60) Provisional application No. 62/887,807, filed on Aug. 16, 2019.

(51) Int. Cl. C07F 15/00

(52) U.S. Cl. CPC C07F 15/0053

(57) ABSTRACT

A process for the preparation of a complex of formula (I): the process comprising the step of reacting a complex of formula (II) or a complex of formula RuX₃.H₂O (IV) with a bidentate ligand of formula (III) wherein R₁, R₂, R₃, R₄, R₅, R₆, R₇, R₈, R₉, R₁₀, A, B, and X are described in the specification; the molar ratio of the complex of formula (II): the bidentate ligand of formula (III) is about 1:6 to about 1:8 or the molar ratio of the complex of formula (IV): the bidentate ligand of formula (III) is about 1:3 to about 1:4; and the process is carried out in water or a water-based solvent, wherein the water-based solvent comprises at least 60% water (by volume) and an organic solvent, at one or more temperatures in the range of about 80° C. to 110° C.

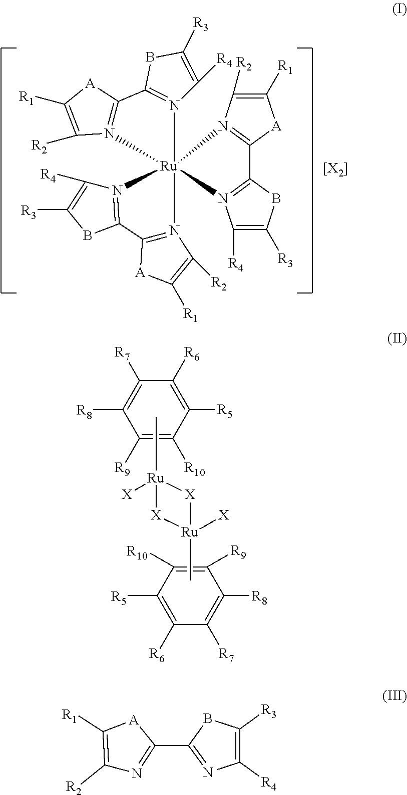

### FIELD

The present invention concerns the preparation of homoleptic ruthenium complexes comprising nitrogen-containing heterocyclic bidentate ligands.

### BACKGROUND

Homoleptic Ru(bipy)₃I₂ (bipy=2,2′-bipyridine) was obtained by heating at reflux RuCl₃3H₂O and 25% excess 2,2-bipyridine (i.e. ratio Ru:NN=1:3.75) in 95% EtOH for 72 h followed by filtration, evaporation, extraction in benzene and precipitation from an aqueous solution of KI (Palmer et al., Inorg. Chem., 1966, 5 (5), 864).

Goss et al. reports the synthesis of homoleptic [Ru(phen-dione)₃](PF₆)₂2H₂O (phen-dione=1,10-phenanthroline-5,6-dione) in a stepwise procedure, by first generating Ru(phen-dione)₂Cl₂ by reaction of RuCl₃.3H₂O with phen-dione in a ratio Ru:NN=1:2, in the presence of LiCl, in DMF at reflux, followed by addition of 1.2 equiv of phen-dione in a 50/50 mixture EtOH/H₂O at reflux. The PF₆ complex was precipitated with saturated aqueous solution of NH₄PF₆. (Inorg. Chem., 1985, 24 (25), 4263).

While the processes described in Palmer et al. and Goss et al. may be used to prepare gram scale quantities of homoleptic Ru(bipy)₃I₂ and [Ru(phen-dione)₃](PF₆)₂2H₂O, the processes are not suitable for large scale manufacture. These processes use RuCl₃.3H₂O as a starting material, the availability of which depends on geographical location. Moreover, these processes use organic and flammable solvents at reflux, as well as toxic solvents such as benzene and DMF, all of which are unsafe on industrial scale. Also, these processes suggest the fact that there is a difference between the ease of adding the three nitrogen-containing heterocyclic bidentate ligands on ruthenium, therefore requiring multi-step synthetic procedures. Various processing steps are also required (e.g. evaporation of solvents, recrystallization, purification) in order to isolate the ruthenium complexes. There is a need to find a one-step process which can accomplish the synthesis of homoleptic ruthenium complexes with three nitrogen-containing heterocyclic bidentate ligands which is suitable on industrial scale.

### SUMMARY OF THE INVENTION

The present invention provides improved processes for the preparation of homoleptic ruthenium complexes with nitrogen-containing heterocyclic bidentate ligands. The processes are suitable for large scale manufacturing. In some embodiments the processes result in high yield. In some embodiments the process results in a product such as [Ru(bpy)₃]Cl₂.6H₂O or [Ru(bpy)₃][PF₆]₂ containing few impurities. In some embodiments, the product is obtained pure as analysed by NMR and/or elemental analysis.

In one aspect, the invention provides a process for the preparation of a complex of formula (I):

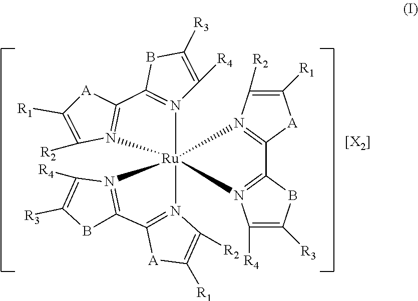

wherein R₁, R₂, R₃ and R₄ are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;

A is selected from the group consisting of:—CRₐR_{b}—, —NRₐ—, O, S, —CRₐ═CR_{b}—, —CRₐ═N—;

B is selected from the group consisting of:—CR_{c}R_{d}—, —NR_{c}—, O, S, —CR_{c}═CR_{d}—, —CR_{c}═N—;

Rₐ, R_{b}, R_{c} and R_{d} are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;

or Rₐ and one of R_{c} and R_{d} or R_{b} and one of R_{c} and R_{d} together with the atoms to which they are bound, form a ring; and

X is a halide;

the process comprising the step of reacting a complex of formula (II)

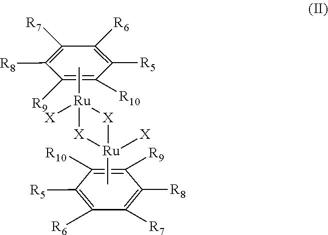

wherein R₅, R₆, R₇, R₈, R₉ and R₁₀ are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl; X is as hereinbefore defined;

with a bidentate ligand of formula (III)

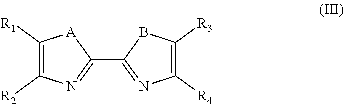

where R₁, R₂, R₃ and R₄, A and B are as hereinbefore defined; wherein the molar ratio of the complex of formula (II): the bidentate ligand of formula (III) is about 1:6 to about 1:8,

characterised in that the process is carried out in water or a water-based solvent, wherein the water-based solvent comprises at least 60% water (by volume) and an organic solvent, at one or more temperatures in the range of about 80° C. to 110° C.

A further aspect of the invention provides a process for the preparation of a compound of formula (I) as hereinbefore defined, the process comprising reacting a compound of formula (IV) RuX₃.H₂O, wherein X is as hereinbefore defined, with a bidentate ligand of formula (III) as hereinbefore defined, wherein the molar ratio of the complex of formula IV: the bidentate ligand of formula (III) is about 1:3 to about 1:4, characterised in that the process is carried out in water or a water-based solvent, wherein the water-based solvent comprises at least 60% water (by volume) and an organic solvent, at one or more temperatures in the range of about 80° C. to 110° C.

Definitions

The point of attachment of a moiety or substituent is represented by “—”. For example, —OH is attached through the oxygen atom.

As used herein, when A is “—CRₐ═N—”, this moiety can be inserted into the complex of formula (I) or the ligand of formula (III) in either order, i.e. as “—CRₐ═N—” or “—N═CRₐ—”.

As used herein, when B is “—CR_{c}═N—”, this moiety can be inserted into the complex of formula (I) or the ligand of formula (III) in either order, i.e. as “—CR_{c}═N—” or “—N═CR_{c}—”.

“Alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like.

The term “cycloalkyl” is used to denote a saturated carbocyclic hydrocarbon radical. The cycloalkyl group may have a single ring or multiple condensed rings. In certain embodiments, the cycloalkyl group may have from 3-20 carbon atoms, in certain embodiments, from 3-10 carbon atoms, in certain embodiments, from 3-8 carbon atoms. The cycloalkyl group may be unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl and the like.

“Alkoxy” refers to an optionally substituted group of the formula alkyl-O— or cycloalkyl-O—, wherein alkyl and cycloalkyl are as defined above.

“Aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

“Arylalkyl” refers to an optionally substituted group of the formula aryl-alkyl-, where aryl and alkyl are as defined above.

“Halide” refers to —F, —Cl, —Br and —I.

“Heteroalkyl” refers to a straight-chain or branched saturated hydrocarbon group wherein one or more carbon atoms are independently replaced with one or more heteroatoms (e.g. nitrogen, oxygen, phosphorus and/or sulfur atoms). The heteroalkyl group may be unsubstituted. Alternatively, the heteroalkyl group may be substituted. Unless otherwise specified, the heteroalkyl group may be attached at any suitable atom and, if substituted, may be substituted at any suitable atom. Examples of heteroalkyl groups include but are not limited to ethers, thioethers, primary amines, secondary amines, tertiary amines and the like.

“Heterocycloalkyl” refers to a saturated cyclic hydrocarbon group wherein one or more carbon atoms are independently replaced with one or more heteroatoms (e.g. nitrogen, oxygen, phosphorus and/or sulfur atoms). The heterocycloalkyl group may be unsubstituted. Alternatively, the heterocycloalkyl group may be substituted. Unless otherwise specified, the heterocycloalkyl group may be attached at any suitable atom and, if substituted, may be substituted at any suitable atom. Examples of heterocycloalkyl groups include but are not limited to epoxide, morpholinyl, piperadinyl, piperazinyl, thirranyl, pyrrolidinyl, pyrazolidinyl, imidazolidinyl, thiazolidinyl, thiomorpholinyl and the like.

“Heteroaryl” refers to an aromatic carbocyclic group wherein one or more carbon atoms are independently replaced with one or more heteroatoms (e.g. nitrogen, oxygen, phosphorus and/or sulfur atoms). The heteroaryl group may be unsubstituted. Alternatively, the heteroaryl group may be substituted. Unless otherwise specified, the heteroaryl group may be attached at any suitable atom and, if substituted, may be substituted at any suitable atom. Examples of heteroaryl groups include but are not limited to thienyl, furanyl, pyrrolyl, imidazolyl, pyrazolyl, thiazolyl, isothiazolyl, oxazolyl, isoxazolyl, triazolyl, thiadiazolyl, thiophenyl, oxadiazolyl, pyridinyl, pyrimidyl, benzoxazolyl, benzthiazolyl, benzimidazolyl, indolyl, quinolinyl and the like.

“Substituted” refers to a group in which one or more hydrogen atoms are each independently replaced with substituents (e.g. 1, 2, 3, 4, 5 or more) which may be the same or different. Examples of substituents include but are not limited to -halo, —C(halo)₃, —R^{m}, ═O, ═S, —O—R^{m}, —S—R^{m}, —NR^{m}Rⁿ, —CN, —NO₂, —C(O)—R^{m}, —COOR^{m}, —C(S)—R^{m}, —C(S)OR^{m}, —S(O)₂OH, —S(O)₂—R^{m}, —S(O)₂NR^{m}Rⁿ, —O—S(O)—R^{m} and —CONR^{m}Rⁿ, such as -halo, —C(halo)₃ (e.g. —CF₃), —R^{m}, —O—R^{m}, —NR^{m}Rⁿ, —CN, or —NO₂. R^{m} and Rⁿ are independently selected from the groups consisting of H, C₁₋₂₀-alkyl, C₆₋₂₀-aryl, C₇₋₂₀-arylalkyl, C₁₋₂₀-heteroalkyl, C₄₋₂₀-heteroaryl, or R^{m} and Rⁿ together with the atom to which they are attached form a heterocycloalkyl group. R^{m} and Rⁿ may be unsubstituted or further substituted as defined herein.

“Bidentate ligands” are ligands that donate two pairs of electrons to a metal atom.

A water-based solvent is a solvent comprising water and an organic solvent, wherein the volume percentage of water is at least 60%.

A non-coordinating anion is an anion that interacts weakly with cations.

As used herein, the abbreviations “bipy” and “bpy” are used interchangeably to refer to 2,2′-bipyridine.

### DETAILED DESCRIPTION

In one aspect, the invention provides a process for the preparation of a complex of formula (I):

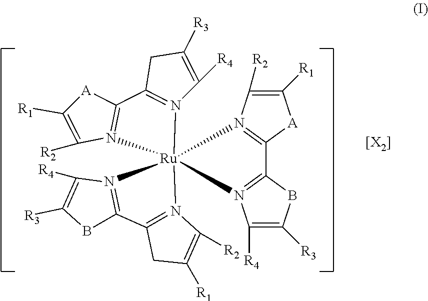

the process comprising the step of reacting a complex of formula (II)

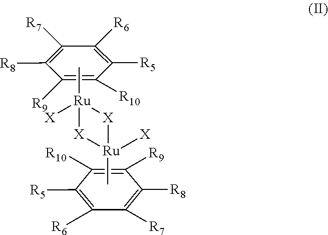

with a bidentate ligand of formula (III)

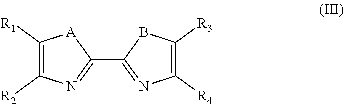

wherein the molar ratio of the complex of formula (II): the bidentate ligand of formula (III) is about 1:6 to about 1:8,
characterised in that the process is carried out in water or a water-based solvent, wherein the water-based solvent comprises at least 60% water (by volume) and an organic solvent, at one or more temperatures in the range of about 80° C. to 110° C.

A further aspect of the invention provides a process for the preparation of a compound of formula (I) as hereinbefore defined, the process comprising reacting a compound of formula RuX₃.H₂O (IV), wherein X is as hereinbefore defined, with a bidentate ligand of formula (III) as hereinbefore defined, wherein the molar ratio of the complex of formula IV: the bidentate ligand of formula (III) is about 1:3 to about 1:4, characterised in that the process is carried out in water or a water-based solvent, wherein the water-based solvent comprises at least 60% water (by volume) and an organic solvent, at one or more temperatures in the range of about 80° C. to 110° C.

The substituents R₁, R₂, R₃ and R₄ are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl.

In one embodiment, R₁, R₂, R₃ and R₄ are independently selected from the group consisting of H, unsubstituted branched or straight chain C₁₋₂₀-alkyl, substituted branched or straight chain C₁₋₂₀-alkyl, unsubstituted C₆₋₂₀-aryl or substituted C₆₋₂₀-aryl.

For example, R₁, R₂, R₃ and R₄ are independently selected from H, branched- or straight-chain alkyl groups (such as methyl, ethyl, n-propyl, iso-propyl, n-butyl, iso-butyl, sec-butyl, tert-butyl, pentyl, hexyl, heptyl, octyl, nonyl, decyl, dodecyl, stearyl), aryl groups (such as phenyl, naphthyl and anthracyl),

In one embodiment, the alkyl groups may be optionally substituted with one or more (e.g. 1, 2, 3, 4, or 5, the number of substituents being dependent on the number of substitutable H atoms) substituents each of which may be the same or different such as halide (F, Cl, Br or I) or alkoxy groups, (e.g. methoxy, ethoxy or propoxy). The aryl group may be optionally substituted with one or more (e.g. 1, 2, 3, 4, or 5, the number of substituents being dependent on the number of substitutable H atoms) substituents each of which may be the same or different such as halide (F, Cl, Br or I), straight- or branched-chain alkyl (e.g. C₁-C₁₀), alkoxy (e.g. C₁-C₁₀alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl.

In one embodiment, R₁ and R₃ are the same.

In another embodiment, R₂ and R₄ are the same.

In yet another embodiment, R₁ and R₃ are the same and R₂ and R₄ are the same.

In one embodiment, R₁, R₂, R₃ and R₄ are the same.

In one embodiment, each of R₁, R₂, R₃ and R₄ are H.

A is independently selected from the group consisting of:—CRₐR_{b}—, —NRₐ—, O, S, —CRₐ═CR_{b}— and —CRₐ═N—; preferably —CRₐ═CR_{b}— or —CRₐ═N—.

B is independently selected from the group consisting of:—CR_{c}R_{d}—, —NR_{c}—, O, S, —CR_{c}═CR_{d}— and —CR_{c}═N—; preferably —CR_{c}═CR_{d}— or —CR_{c}═N—.

In one embodiment, A and B are the same.

In one embodiment, A and B are —CRₐ═CR_{b}— and —CR_{c}═CR_{d}— respectively.

In one embodiment, A and B are each —CH═CH—.

Rₐ, R_{b}, R_{c} and R_{d} are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;

or Rₐ and one of R_{c} and R_{d} or R_{b} and one of R_{c} and R_{d} together with the atoms to which they are bound, form a ring, suitably a 6-membered ring.

Rₐ, R_{b}, R_{c} and R_{d} may independently be H, unsubstituted branched- or straight-chain alkyl groups (such as methyl, ethyl, n-propyl, iso-propyl, n-butyl, iso-butyl, sec-butyl, tert-butyl, pentyl, hexyl, heptyl, octyl, nonyl, decyl, dodecyl, stearyl), aryl groups (such as phenyl, naphthyl and anthracyl), In another embodiment, the alkyl groups may be optionally substituted with one or more (e.g. 1, 2, 3, 4, or 5, the number of substituents being dependent on the number of substitutable H atoms) substituents such as halide (—F, —Cl, —Br or —I) or alkoxy groups (e.g. methoxy, ethoxy or propoxy). The aryl group may be optionally substituted with one or more (e.g. 1, 2, 3, 4, or 5, the number of substituents being dependent on the number of substitutable H atoms) substituents such as halide (—F, —Cl, —Br or —I), straight- or branched-chain C₁-C₁₀-alkyl, C₁-C₁₀alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—).

In one embodiment, Rₐ is H and R_{b} is selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl.

In one embodiment, Rₐ is methoxy and R_{b} is selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl.

In another embodiment, R_{c} is H and R_{d} is selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl.

In another embodiment, R_{c} is methoxy and R_{d} is selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl.

In one embodiment, Rₐ and R_{c} are each H; and R_{b} and R_{d} are the same and are selected from unsubstituted branched- or straight-chain alkyl groups (such as methyl, ethyl, n-propyl, iso-propyl, n-butyl, iso-butyl, sec-butyl, tert-butyl, pentyl, hexyl, heptyl, octyl, nonyl, decyl, dodecyl, stearyl), aryl groups (such as phenyl, naphthyl and anthracyl).

In one embodiment, Rₐ and R_{c} are each methoxy; and R_{b} and R_{d} are the same and are selected from unsubstituted branched- or straight-chain alkyl groups (such as methyl, ethyl, n-propyl, iso-propyl, n-butyl, iso-butyl, sec-butyl, tert-butyl, pentyl, hexyl, heptyl, octyl, nonyl, decyl, dodecyl, stearyl), aryl groups (such as phenyl, naphthyl and anthracyl).

In an alternative embodiment, Rₐ and R_{c} are each H; and R_{b} and R_{d} are the same and are selected from substituted branched- or straight-chain alkyl groups or substituted aryl groups. The alkyl groups may be optionally substituted with one or more (e.g. 1, 2, 3, 4, or 5, the number of substituents being dependent on the number of substitutable H atoms) substituents such as halide (—F, —Cl, —Br or —I) or alkoxy groups,(e.g. methoxy, ethoxy or propoxy). The aryl group may be optionally substituted with one or more (e.g. 1, 2, 3, 4, or 5, the number of substituents being dependent on the number of substitutable H atoms) substituents such as halide (—F, —Cl, —Br or —I), straight- or branched-chain C₁-C₁₀-alkyl, C₁-C₁₀alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—).

In an alternative embodiment, Rₐ and R_{c} are each methoxy; and R_{b} and R_{d} are the same and are selected from substituted branched- or straight-chain alkyl groups or substituted aryl groups. The alkyl groups may be optionally substituted with one or more (e.g. 1, 2, 3, 4, or 5, the number of substituents being dependent on the number of substitutable H atoms) substituents such as halide (—F, —Cl, —Br or —I) or alkoxy groups,(e.g. methoxy, ethoxy or propoxy). The aryl group may be optionally substituted with one or more (e.g. 1, 2, 3, 4, or 5, the number of substituents being dependent on the number of substitutable H atoms) substituents such as halide (—F, —Cl, —Br or —I), straight- or branched-chain C₁-C₁₀-alkyl, C₁-C₁₀alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—).

In one embodiment, each of R₁, R₂, R₃ and R₄ are H; A and B are —CRₐ═CR_{b}— and —CR_{c}═CR_{d} respectively; Rₐ and R_{c} are H; R_{b} and R_{d} are the same and are H, CH₃, t-Bu or CF₃. In a preferred embodiment, R_{b} and R_{d} are hydrogen.

Preferably the ligand in the complex of formula (I) and the ligand of formula (III) is:

bipy=2,2′-bipyridine

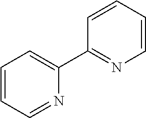 [SMILES: c1ccc(-c2ccccn2)nc1]

dmbpy=4,4′-Bis(methyl)-2,2′-bipyridine (which is also known as 2,2′-bi-4-picoline);

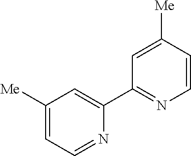 [SMILES: Cc1ccnc(-c2cc(C)ccn2)c1]

dtbbpy=4,4′-Bis(tert-butyl)-2,2′-bipyridine;

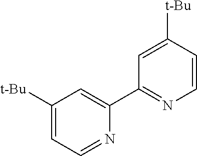 [SMILES: CC(C)(C)c1ccnc(-c2cc(C(C)(C)C)ccn2)c1]

4,4′-btfmb=4,4′-Bis(trifluoromethyl)-2,2′-bipyridine;

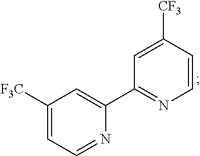 [SMILES: FC(F)(F)c1ccnc(-c2cc(C(F)(F)F)ccn2)c1]

or

5,5′-btfmb=5,5′-Bis(trifluoromethyl)-2,2′-bipyridine;

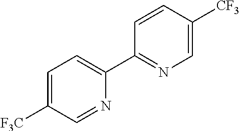 [SMILES: FC(F)(F)c1ccc(-c2ccc(C(F)(F)F)cn2)nc1]

In an alternative embodiment, A and B are —CRₐ═CR_{b}— and —CR_{c}═CR_{d} respectively and Rₐ and one of R_{c} and R_{d} or R_{b} and one of R_{c} and R_{d} together with the atoms to which they are bound, form a ring, suitably a 6-membered ring; optionally, the ring is aromatic. For example, Rₐ and R_{c} or R_{d}, together with the atoms to which they are bound, form a ring, suitably a 6-membered ring. Alternatively, R_{b} and R_{c} or R_{d}, together with the atoms to which they are bound, form a ring, suitably a 6-membered ring.

Suitably, each of R₁, R₂, R₃ and R₄ are H; A is —CH═CR_{b}—; B is —CH═CR_{d}—; R_{b} and R_{d} together with the carbon atoms to which they are bound form a ring; suitably a 6-membered ring.

Preferably, the ligand in the complex of formula (I) and the ligand of formula (III) is:

phen=1,10-phenanthroline.

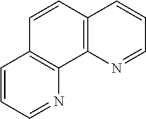 [SMILES: c1cnc2c(c1)ccc1cccnc12]

Suitably, each of R₁, R₂, R₃ and R₄ are H; A is —CRₐ═CR_{b}—; B is —CR_{c}═CR_{d}—; R_{b} and R_{d} together with the carbon atoms to which they are bound form a ring, suitably a 6-membered ring.

Suitably, Rₐ and R_{c} are methoxy groups and R_{b} and R_{c} together with the carbon atoms to which they are bound form a ring, suitably a 6-membered ring.

Preferably, the ligand in the complex of formula (I) and the ligand of formula (III) is:

OMe-phen=4,7-dimethoxy-1,10-phenanthroline

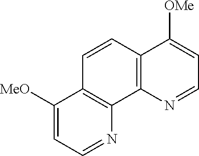 [SMILES: COc1ccnc2c1ccc1c(OC)ccnc12]

In an alternative embodiment, A and B are the same and are —CRₐ═N— and —CR_{c}═N— respectively. In a preferred embodiment, Rₐ and R_{c} are H. In a most preferred embodiment, each of R₁, R₂, R₃ and R₄ are H; and Rₐ and R_{c} are H.

Preferably the ligand in the complex of formula (I) and the ligand of formula (III) is:

bpz=2,2′-bipyrazine

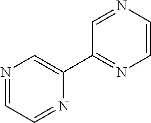 [SMILES: c1cnc(-c2cnccn2)cn1]

Preferably the ligand in the complex of formula (I) and the ligand of formula (III) is:

bpm=2,2′-bipyrimidine

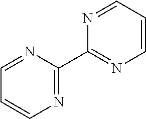 [SMILES: c1cnc(-c2ncccn2)nc1]

Halide X may be fluoride, chloride, bromide or iodide. Preferably, the halide is chloride.

The complex of formula (I) may be:

(i) Ru(bipy)₃Cl₂,

(ii) Ru(dmbpy)₃Cl₂,

(iii) Ru(dtbbpy)₃Cl₂,

(iv) Ru(4,4′-btfmb)₃Cl₂,

(v) Ru(5,5′-btfmb)₃Cl₂,

(vi) Ru(bpz)₃Cl₂,

(vii) Ru(1,10-phen)₃Cl₂,

(viii) Ru(OMe-phen)₃Cl₂,

(ix) Ru(bpm)₃Cl₂.

In a most preferred embodiment, the complex of formula (I) is Ru(bipy)₃Cl₂.

R₅, R₆, R₇, R₈, R₉ and R₁₀ in the complexes of formula (II) may be independently selected from the group consisting H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl.

R₅, R₆, R₇, R₈, R₉ and R₁₀ may independently be H, branched- or straight-chain alkyl groups (such as methyl, ethyl, n-propyl, iso-propyl, n-butyl, iso-butyl, sec-butyl, tert-butyl, pentyl (e.g. n-pentyl or neopentyl), hexyl, heptyl, octyl, nonyl, decyl, dodecyl or stearyl), cycloalkyl groups (such as cyclopropyl, cyclobutyl, cyclopentyl, cyclohexyl or adamantly), aryl groups (such as phenyl, naphthyl or anthracyl).

In one embodiment, the alkyl groups may be optionally substituted with one or more (e.g. 1, 2, 3, 4, or 5, the number of substituents being dependent on the number of substitutable H atoms) substituents each of which may be the same or different such as halide (F, Cl, Br or I) or alkoxy groups, e.g. methoxy, ethoxy or propoxy. The aryl group may be optionally substituted with one or more (e.g. 1, 2, 3, 4, or 5, the number of substituents being dependent on the number of substitutable H atoms) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl.

In one embodiment, R₅, R₆, R₇, R₈, R₉ and R₁₀ are the same. Preferably, each of R₅, R₆, R₇, R₈, R₉ and R₁₀ are H.

In another embodiment, at least one of R₅, R₆, R₇, R₈, R₉ and R₁₀ is selected from a group which is not —H. For example, one of R₅, R₆, R₇, R₈, R₉ and R₁₀ may be selected from a group which is not —H, such as two of R₅, R₆, R₇, R₈, R₉ and R₁₀, three of R₅, R₆, R₇, R₈, R₉ and R₁₀, four of R₅, R₆, R₇, R₈, R₉ and R₁₀, five of R₅, R₆, R₇, R₈, R₉ and R₁₀ or all of R₅, R₆, R₇, R₈, R₉ and R₁₀.

In another embodiment, five of R₅, R₆, R₇, R₈, R₉ and R₁₀ are —H, and the other one of R₅, R₆, R₇, R₈, R₉ and R₁₀ is selected from the group consisting of halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl and substituted C₆₋₂₀-aryl. In a preferred embodiment, five of R₅, R₆, R₇, R₈, R₉ and R₁₀ are —H, and the other one of R₅, R₆, R₇, R₈, R₉ and R₁₀ is a branched- or straight-chain alkyl. In another embodiment, five of R₅, R₆, R₇, R₈, R₉ and R₁₀ are —H (e.g. R₆, R₇, R₈, R₉ and R₁₀), and the other one of R₅, R₆, R₇, R₈, R₉ and R₁₀ (e.g. R₅) is selected from the group consisting of C₁₋₅-alkyl, such as -Me, -Et, -Pr (n- or i-), -Bu (n-, i- or t-), for example, -Me, -iPr.

In another embodiment, four of R₅, R₆, R₇, R₈, R₉ and R₁₀ are —H, and the other two of R₅, R₆, R₇, R₈, R₉ and R₁₀ re independently selected from the group consisting of halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl and substituted C₆₋₂₀-aryl. In a preferred embodiment, four of R₅, R₆, R₇, R₈, R₉ and R₁₀ are —H, and the other two of R₅, R₆, R₇, R₈, R₉ and R₁₀ are independently selected from the group consisting of branched- or straight-chain alkyl. In another embodiment, four of R₅, R₆, R₇, R₈, R₉ and R₁₀ are —H (e.g. R₆, R₇, R₉ and R₁₀), and the other two of R₅, R₆, R₇, R₈, R₉ and R₁₀ (e.g. R₅ and R₈) are independently selected from the group consisting of C₁₋₅-alkyl, such as -Me, -Et, -Pr (n- or i-), -Bu (n-, i- or t-), for example, -Me, -iPr.

In another embodiment, three of R₅, R₆, R₇, R₈, R₉ and R₁₀ are —H, and the other three of R₅, R₆, R₇, R₈, R₉ and R₁₀ are independently selected from the group consisting of halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl and substituted C₆₋₂₀-aryl. In a preferred embodiment, three of R₅, R₆, R₇, R₈, R₉ and R₁₀ are —H, and the other three of R₅, R₆, R₇, R₈, R₉ and R₁₀ are independently selected from the group consisting of branched- or straight-chain alkyl. In another embodiment, three of R₅, R₆, R₇, R₈, R₉ and R₁₀ are —H (e.g. R₆, R₈ and R₁₀), and the other three of R₅, R₆, R₇, R₈, R₉ and R₁₀ (e.g. R₅, R₇ and R₉) are independently selected from the group consisting of C₁₋₅-alkyl, such as -Me, -Et, -Pr (n- or i-), -Bu (n-, i- or t-), for example, -Me, -iPr.

In another embodiment, two of R₅, R₆, R₇, R₈, R₉ and R₁₀ are —H, and the other four of R₅, R₆, R₇, R₈, R₉ and R₁₀ are independently selected from the group consisting of halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl and substituted C₆₋₂₀-aryl. In a preferred embodiment, two of R₅, R₆, R₇, R₈, R₉ and R₁₀ are —H, and the other four R₅, R₆, R₇, R₈, R₉ and R₁₀ are independently selected from the group consisting of branched- or straight-chain alkyl. In another embodiment two of R₅, R₆, R₇, R₈, R₉ and R₁₀ are —H (e.g. R₅ and R₈), and the other four of R₅, R₆, R₇, R₈, R₉ and R₁₀ (e.g. R₆, R₇, R₉ and R₁₀) are independently selected from the group consisting of C₁₋₅-alkyl, such as -Me, -Et, —Pr (n- or i-), -Bu (n-, i- or t-), for example, -Me, -iPr.

In another embodiment, one of R₅, R₆, R₇, R₈, R₉ and R₁₀ is —H and the other five of R₅, R₆, R₇, R₈, R₉ and R₁₀ are independently selected from the group consisting of halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl and substituted C₆₋₂₀-aryl. In a preferred embodiment, one of R₅, R₆, R₇, R₈, R₉ and R₁₀ is —H and the other five of R₅, R₆, R₇, R₈, R₉ and R₁₀ are independently selected from the group consisting of branched- or straight-chain alkyl. In another embodiment, one of R₅, R₆, R₇, R₈, R₉ and R₁₀ is —H (e.g. R₅) and the other five of R₅, R₆, R₇, R₈, R₉ and R₁₀ (e.g. R₆, R₇, R₈, R₉ and R₁₀) are selected from the group consisting of C₁₋₅-alkyl, such as -Me, -Et, —Pr (n- or i-), -Bu (n-, i- or t-), for example, -Me, -iPr.

X is as hereinbefore defined for complexes of formula (I).

In one embodiment, the complex of formula (II) is [{RuCl₂(benzene)}₂.

In another embodiment, the complex of formula (II) is [{RuCl₂(p-cymene)}₂.

In another embodiment, the complex of formula (II) is [{RuCl₂(mesitylene)}₂.

The process uses commercially available starting material complexes of formula (II) and (IV) and bidentate ligands of formula (III) which may readily be made following literature methods.

The complexes of formula (II) or of formula (IV) and the bidentate ligand of formula (III) are mixed together in water or a water-based solvent.

In one embodiment, the complexes of formula (II) or of formula (IV) and the bidentate ligand of formula (III) are mixed together in water.

In an alternative embodiment, the complexes of formula (II) or of formula (IV) and the bidentate ligand of formula (III) are mixed together in a water-based solvent, wherein the water-based solvent is a mixture of water and an organic solvent, wherein the water content is at least 60% (volume). Preferably, the organic solvent is an alcohol or an ether. Suitable alcohols are methanol (MeOH), ethanol (EtOH), n-propanol (nPrOH), iso-propanol (iPrOH) and t-amyl alcohol (t-amyIOH), preferably methanol (MeOH), ethanol (EtOH), n-propanol (nPrOH) and iso-propanol (iPrOH). Suitable ethers are tetrahydrofuran (THF), 2-methyltetrahydrofuran (2-Me-THF), 3-methyltetrahydrofuran (3-Me-THF) and dioxane; particularly THF. A particularly preferred organic solvent is ethanol.

In an embodiment, the water content of the water-based solvent is at least 60%, at least 65%, at least 70%, at least 75%, at least 80%, at least 85%, at least 90% or at least 95%. Preferably, the water content of the water-based solvent is at least 90% or at least 95%.

The concentration of the complexes of formula (II) or of formula (IV) in the water-based solvent is about 0.005 mmol/mL to about 5 mmol/mL, preferably about 0.01 mmol/mL to about 2.5 mmol/mL, even more preferably 0.1 mmol/mL to 1 mmol/mL.

In the present invention, the molar ratio of the complex of formula (II): the bidentate ligand of formula (III) is about 1:6 to about 1:8 or the molar ratio of the complex of formula (IV): the bidentate ligand of formula (III) is about 1:3 to about 1:4. The molar ratio of the complex of formula (II): the bidentate ligand of formula (III) may be 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, preferably 1:6.0, 1:6.1, 1:6.2, 1:6.3, 1:6.4, 1:6.5; more preferably 1:6.0 or 1:6.1. The molar ratio of the complex of formula (IV): the bidentate ligand of formula (III) may be 1:3.0, 1:3.1, 1:3.2, 1:3.3, 1:3.4, 1:3.5, 1:3.6, 1:3.7, 1:3.8, 1:3.9, 1:4.0; preferably 1:3.0, 1:3.1, 1:3.2; more preferably 1:3.0.

In reacting the complexes of formula (II) or of formula (IV) and the bidentate ligand of formula (III) in water or the water-based solvent, the components may be mixed in any suitable order, although preferably the complex of formula (II) or of formula (IV) is first added to water or the water-based solvent, followed by the bidentate ligand of formula (III).

After the complexes of formula (II) or of formula (IV) and the bidentate ligand of formula (III) are mixed together in water or the water-based solvent, preferably the reaction mixture is stirred at a temperature in the range of about 80° C. to about 110° C., suitably about 85° C. to about 110° C., suitably about 90° C. to about 110° C., preferably about 95° C. to about 105° C., even more preferably at 100° C.

The mixture may be stirred for a period e.g. preferably about 30 minutes to about 72 hours, more preferably about 5 hours to about 24 hours, more preferably 10 hours to 20 hours and most preferably about 16 hours.

On completion of the reaction, the complex of formula (I) may be separated from the reaction mixture by any appropriate method which is dependent on the physical form of the product, optionally with the aid of an anti-solvent, such as acetone, methyl tert-butyl ether (MTBE). For example, when it is desired to recover the complex of formula (I) as a solid, the complex may be isolated from the reaction mixture by distillation, filtration, decanting or centrifuging. The separated complex is preferably washed with further solvent and then dried. Drying may be performed using known methods, for example, at temperatures in the range of about 10-60° C. and preferably about 20-40° C. under about 1-30 mbar vacuum for about 1 hour to about 5 days.

In a further embodiment, the present invention provides a process for the preparation of a compound of formula (V),

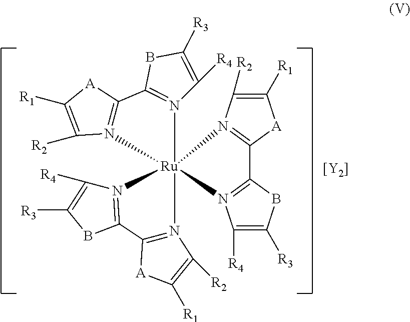

wherein R₁, R₂, R₃, R₄, A and B are as hereinbefore defined; and

Y is a non-coordinating anion or a halide which is different to X as defined in relation to the complex of formula (I);

said process comprising reacting a complex of formula (I) as hereinbefore defined with a compound of formula RY, wherein R is selected from a group consisting of an alkali metal cation, Ag⁺ and a quaternary ammonium cation and Y is as hereinbefore defined, in a molar ratio of complex of formula (I): RY of at least 1:2 and at most 1:3, characterized in that the process is carried out in water or a water-based solvent, wherein the water-based solvent comprises at least 60% water (volume) and an organic solvent, at one or more temperatures in the range of about 1000 to 50° C.

R is suitably K⁺, Na⁺, Ag⁺ or [R′₄N]⁺, wherein R′ is H or an alkyl.

Y is suitably PF₆⁻, BF₄⁻, BPh₄⁻, SbF₆⁻, [{3,5-(CF₃)₂C₆H₃}₄B]⁻ ([BAr^{F}₄]⁻), CF₃SO₃⁻ (OTf), ArFSO₃⁻, [(CF₃SO₂)₂N]⁻ (TFSI), F, Cl, Br or I.

Examples of suitable compounds of formula RY include NaI, tetra-n-butylammonium iodide, NaPF₆, KPF₆, AgPF₆, NaBF₄, KBF₄, NaBAr^{F}₄, Preferably, the compound of formula RY is KPF₆ or AgPF₆. Most preferably, the compound of formula RY is KPF₆.

Preferably, the compounds of formula (V) are:

[Ru(bipy)₃]Y₂;

[Ru(dmbpy)₃]Y₂;

[Ru(dtbbpy)₃]Y₂;

[Ru(4,4′-btfmb)₃]Y₂;

[Ru(5,5′-btfmb)₃]Y₂;

[Ru(bpz)₃]Y₂;

[Ru(phen)₃]Y₂;

[Ru(OMe-phen)₃]Y₂;

[Ru(bpm)₃]Y₂.

In a most preferred embodiment, the complex of formula (V) is Ru(bipy)₃(PF₆)₂.

In another most preferred embodiment, the complex of formula (V) is Ru(1,10-phenanthroline)₃(PF₆)₂.

In another most preferred embodiment, the complex of formula (V) is Ru(dmbpy)₃(PF₆)₂.

In another most preferred embodiment, the complex of formula (V) is Ru(bpm)₃(PF₆)₂.

The molar ratio of complex of formula (I): RY may be 1:2.0, 1:2.1, 1:2.2, 1:2.3, 1:2.4, 1:2.5, 1:2.6, 1:2.7, 1:2.8, 1:2.9 or 1:3.0. Preferably, the molar ratio of complex of formula (I): RY is 1:2.0 or 1:2.1.

The components may be combined in any suitable order, although it is preferred that the complex of formula (I) in water or a water-based solvent is combined with the compound of formula RY in water or a water-based solvent.

The water-based solvent is generally as described above.

The process of the invention may be carried out at one or more temperatures in the range of about 10° C. to about 50° C., preferably about 15° C. to about 30° C., for example, about 20° C. to about 25° C.

In one embodiment, the complex of formula (V) is prepared without prior isolation of the complex of formula (I).

The complex of formula (V) may then be isolated as generally described above in relation to complexes of formula (I).

Compounds of formula (I) or (V) show particular utility as photoredox catalysts for carbon-carbon or carbon-heteroatom bond formation in the synthesis of pharmaceutical and agrochemical compounds, as described for example in Org. Process. Res. Dev. 2016, 20, 1134-1147.

The invention will be further illustrated by reference to the following non-limiting Examples.

### EXAMPLES

### General Information

All reactions were carried out under a nitrogen atmosphere in solvent using commercially available reagents that were purchased and used as received. No attention was paid to the drying of solvents. 1,10-phenanthroline was purchased from Sigma Aldrich and used as received. 2,2′-Bi-4-picoline (dmbpy) and 2,2′-bipyrimidine were purchased from Oakwood Chemicals and used as received. For kilogram scale reactions, 2,2′-bipridine and KPF₆ were purchased from RennoTech and used as received. [Ru(Cl)₂(p-cymene)]₂ was supplied by Johnson Matthey. Kilogram scale experiments were conducted in a Chemglass 30 L jacketed reactor using (Huber Unistat 510) for heating and cooling. All ¹H NMR, ¹³C NMR, ³¹P NMR and ¹⁹F NMR spectra were recorded on a Bruker Avance DRX-400 spectrometer at ambient temperature; chemical shifts (δ) are given in ppm. ¹H and ¹³C NMR spectra were referenced to the NMR solvent peaks or internal TMS. ³¹P NMR spectra were calibrated to an external phosphoric acid standard (85% in D₂O as provided by Sigma Aldrich). Coupling constants (J) are reported in Hz and apparent splitting patterns are designated using the following abbreviations: s (singlet), d (doublet), t (triplet), q (quartet), sept (septet), m (multiplet), br (broad), app. (apparent) and the appropriate combinations. All reactions were carried out under a nitrogen atmosphere. The identity of known isolated products was confirmed by comparison with literature spectroscopic data. The purity of the isolated products was >95% as determined by ¹H NMR or elemental analysis.

### Example 1: Conversion to [Ru(Bpy)₃][Cl]₂ with Variation in the EtOH: H₂O Solvent Composition

In EtOH as the only solvent, the reaction proceeded to give only about 5% of desired product [Ru(bpy)₃][Cl]2 (Example 1A). A 10:1 EtOH:H₂O ratio provided [Ru(bpy)₃][Cl]2 in 11% conversion (Example 1B). Increasing the water content first to a 2:1, then to a 1:2 EtOH:H₂O ratio, resulted in improved conversions to [Ru(bpy)₃][Cl]₂ (47 and 97% respectively; Examples 1C and 1D). Further step-wise changes of EtOH: H₂O to 1:2.5 and 1:3 produced desired product [Ru(bpy)₃][Cl]₂ in close to quantitative conversions and 78 and 80% isolated yield, respectively (Examples 1E and 1F). Reducing the ethanol content even further, to a 1:10 EtOH:H₂O ratio improved the isolated yield to 92% (Example 1G). Complete removal of ethanol from the reaction mixture resulted in 100% conversion and 94% isolated yield of [Ru(bpy)₃][Cl]₂ (Example 1H). The results are shown in Table 1.

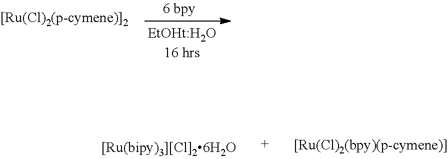

TABLE 1
Conversion to [Ru(bpy)₃][Cl]₂ with variation
in the EtOH:H₂O solvent composition

| | EtOH:H₂O | | | |
|---|---|---|---|---|
| | ratio | Temp | Conv. to | |
| Example | (by volume) | (° C.) ^{[a]} | [Ru(bpy)₃][Cl]₂ (%) ^{[b]} | |
| 1A ^{[d]} | 1:0 | 80 | 5 | |
| 1B ^{[d]} | 10:1 | 80 | 11 | |
| 1C ^{[d]} | 2:1 | 80 | 47 | |
| 1D | 1:2 | 85 | 97 | |
| 1E | 1:2.5 | 90 | 98 | (78) |
| 1F | 1:3 | 90 | >99 | (80) |
| 1G | 1:10 | 100 | 100 | (92) |
| 1H ^{[c]} | 0:1 | 100 | 100 | (94) |

^{[a]} Reflux of the mixtures was observed at the listed temperatures;
^{[b]} Isolated yield in parentheses;
^{[c]} average of 5 reactions;
^{[d]} not according to the invention

Solvent Screening for the Preparation of [Ru(Bpy)₃][Cl]₂•x H₂O (Examples 2-7)

A 100 mL two-necked round bottom flask equipped with a condenser, nitrogen inlet adapter and Teflon-coated stir bar is charged with the Ru precursor and 2,2′-bipyridine. The flask is sealed, then evacuated and backfilled with nitrogen three times. Solvent is added via syringe, and the reaction is stirred at the indicated temperature for 16 hours. The reaction mixture is cooled to ambient temperature, and anti-solvent is added (if applicable). The resulting mixture is stirred for 30 min, and the solids are isolated by filtration. The solids are washed with the specified solvent and dried in vacuo. The resulting solids are characterized by NMR spectroscopy and elemental analysis in certain cases. The results are shown in Table 2.

Example 2: [Ru(Cl)₂(p-cymene)]2 (0.61 g, 1 mmol); 2,2′-bipyridine (0.94 g, 6.02 mmol); H₂O(4.5 mL) and THF (0.45 mL) 100° C., 16 hrs; acetone (15 mL) as antisolvent; acetone (2×10 mL) to wash final product. Title compound obtained as a bright orange solid (1.27 g, 85%).

Example 3: [Ru(Cl)₂(p-cymene)]2 (0.61 g, 1.00 mmol); 2,2′-bipyridine (0.94 g, 6.02 mmol); H₂O(4.5 mL) and iPrOH (0.45 mL); 100° C., 16 hrs; acetone (15 mL) as antisolvent; acetone (2×10 mL) to wash final product. Title compound obtained as a bright orange solid (1.36 g, 91%).

Example 4: [Ru(Cl)₂(p-cymene)]2 (1.00 g, 1.63 mmol); 2,2′-bipyridine (1.53 g, 9.77 mmol); MeOH (2.7 mL) and H₂O (27 mL); 100° C., 16 hrs; acetone (120 mL) added as antisolvent; acetone (3×10 mL) used to wash final product. Title compound obtained as a bright orange solid (1.51 g, 72%).

Example 5: [Ru(Cl)₂(p-cymene)]2 (1.00 g, 1.63 mmol); 2,2′-bipyridine (1.53 g, 9.77 mmol); EtOH (2.7 mL) and H₂O (27 mL); 100° C., 16 hrs; Title compound obtained as a bright orange solid (2.25 g, 92%).

Example 6: [Ru(Cl)₂(p-cymene)]2 (1.00 g, 1.63 mmol); 2,2′-bipyridine (1.53 g, 9.77 mmol); H₂O(30 mL); 100° C., 16 hrs; THE (150 mL) added as antisolvent; THE (20 mL) used to wash final product. Title compound obtained as a bright orange solid (1.82 g, 87%).

### Example 7: Representative Procedure for Intermediate Scale-Up of Synthesis of [Ru(Bpy)₃][Cl]₂

(Table 2, Entry 6):

A 1 L multi-necked round bottom flask equipped with a condenser, nitrogen inlet adapter and Teflon-coated stir bar was charged with [Ru(Cl)₂(p-cymene)]₂ (125.00 g, 203.46 mmol) and 2,2′-bipyridine (190.66 g, 1.22 mol). The flask was sealed, then evacuated and backfilled with nitrogen three times. Water (320 mL) was added, and the reaction mixture was heated to 100° C. in an oil bath. The mixture was stirred at this temperature for 16 hrs. The reaction mixture was cooled to ambient temperature and transferred to 5 L fishbowl. Acetone (2 L) was added, and the resulting red slurry was stirred for 30 min. The solids were isolated by filtration and washed with acetone (3×300 mL). The product was dried in vacuo to yield the title compound obtained as a bright orange solid (300 g, 99%). Characterisation data consistent with those previously reported in the literature (see, for example, Inorg. Chem. 2008, 47, 14, 6427-6434). ¹H NMR (DMSO-d6, 400 MHz): δ 8.91 (d, J 8.4, 6H), 8.18 (t, J 6.4, 6H), 7.74 (d, J 5.2, 6H), 7.57-7.53 (m, 6H); Anal. Calcd for C₃₀H₃₆Cl₂N₆O₆Ru: C, 48.13; H, 4.85; N, 11.23; Ru, 13.50. Found: C, 47.58; H, 4.49; N, 10.98; Ru, 13.25.

TABLE 2
Preparation of [Ru(bpy)₃][Cl]₂
according to the invention

| | Solvent | Temp | Conv. to |
|---|---|---|---|
| Example | (ratio) | (° C.) | [Ru(bpy)₃][Cl]₂ (%)^{[a]} |
| 2 | H₂O:THF 10:1 | 100 | 100 (85) |
| 3 | H₂O:iPrOH 10:1 | 100 | 100 (91) |
| 4 | H₂O:MeOH 10:1 | 100 | 100 (72) |
| 5 | H₂O:EtOH 10:1 | 100 | 100 (92) |
| 6 ^{[b]} | H₂O | 100 | 100 (87) |
| 7 | H₂O | 100 | 100 (99) |

^{[a]}Isolated yield in parentheses;
^{[b]} THF added as anti-solvent

Procedures for the preparation of [Ru(bpy)₃][PF₆]2

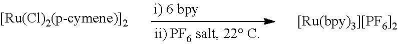

Example 8: Preparation of [Ru(bpy)₃][PF₆]₂ without isolation of intermediate [Ru(bpy)₃][Cl]₂.6H₂O: A 250 mL two-necked round bottom flask was charged with the [Ru(Cl)₂(p-cymene)]2 (5.00 g, 8.14 mmol) and 2,2-bipyridine (7.63 g, 48.83 mmol). The flask was equipped with a condenser attached to a nitrogen inlet and purged with N₂. H₂O (100 mL) was added through the second port of the flask. This was then sealed with a glass stopper, and the reaction stirred at reflux temperature. After 16 hrs, the reaction mixture was allowed to cool to ambient temperature. Then a solution of NH₄PF₆ (2.79 g, 17.1 mmol) in H₂O (50 mL) was added, the flask rinsed with another 30 mL of H₂O, then the orange slurry stirred for 30 min. Then the solid was isolated by filtration and washed with H₂O (2×25 mL) and Et₂O (2×25 mL). The bright orange product was dried in vacuo and weighed (7.29 g). Additional NH₄PF₆ was required, so product was re-combined with mother liquor, a solution of NH₄PF₆ (2.79 g, 17.1 mmol) in H₂O(50 mL) was added under N₂ atmosphere, the flask rinsed with another 30 mL of H₂O, then the orange slurry stirred for 30 min. Then the solid was isolated by filtration and washed with H₂O (3×50 mL) and Et₂O (2×50 mL). The bright orange product was dried in vacuo and weighed (13.60 g, 97%).

### Example 9: Preparation of [Ru(Bpy)₃][PF₆]₂ with Isolation of Intermediate [Ru(Bpy)₃][Cl]₂.6H₂O

A 250 mL two-necked roundbottom flask was charged with the [Ru(Cl)₂(p-cymene)]₂ (10.0 g, 16.33 mmol) and 2,2-bipyridine (15.30 g, 97.98 mmol). The flask was equipped with a condenser attached to a N₂ inlet and purged with N₂. H₂O (50 mL) was added through the second port of the flask. This was then sealed with a glass stopper, and the reaction stirred at reflux temperature. After 16 hrs, the reaction mixture was allowed to cool to ambient temperature. The reaction mixture was then decanted into a 500 mL roundbottom flask, and acetone (300 mL) added. It was stirred for 20 min, then the orange solid was isolated by filtration and washed with acetone (2×50 mL). The resulting solid was dried for 10 min, then charged to a 500 mL roundbottom flask with a magnetic stirrer, and H₂O (200 mL) added to form a red suspension. Then a solution of NH₄PF₆ (11.71 g, 71.85 mml) in H₂O (50 mL) was added, the flask was rinsed with another 50 mL H₂O, and the orange slurry stirred for 1 hour 25 min. Then the solid was isolated by filtration and washed with H₂O (3×100 mL) and Et₂O (2×50 mL). The bright orange product was dried in vacuo and weighed (25.67 g, average yield from 2 reactions: 85%).

Example 10: Preparation of [Ru(bpy)₃][PF₆]₂ without isolation of intermediate [Ru(bpy)₃][Cl]₂.6H₂O: A 250 mL two-necked round bottom flask was charged with the [Ru(Cl)₂(p-cymene)]2 (5.00 g, 8.14 mmol) and 2,2-bipyridine (7.63 g, 48.83 mmol). The flask was equipped with a condenser attached to a nitrogen inlet and purged with N₂. H₂O (100 mL) was added, the flask was sealed with a glass stopper, and the reaction mixture stirred at 100° C. After 16 hrs, the reaction mixture was cooled to ambient temperature. A solution of KPF₆ (6.59 g, 35.81 mmol) in H₂O (60 mL) was added and the orange slurry stirred for 1 hour. The solids were isolated by filtration and washed with H₂O (3×150 mL). The product was dried in vacuo to give the title compound as a bright orange solid (13.78 g, 98%)

### Example 11: Preparation of [Ru(Bpy)₃][PF₆]₂ with Isolation of Intermediate [Ru(Bpy)₃][Cl]₂•6H₂O

A 1000 mL two-necked round bottom flask was charged with the [Ru(Cl)₂(p-cymene)]₂ (125 g, 203.46 mmol) and 2,2-bipyridine (190.66 g, 1.221 mol). The flask was equipped with a condenser attached to a nitrogen inlet and purged with nitrogen. H₂O (320 mL) was added through the second port of the flask. This was then sealed with a glass stopper, and the reaction stirred at reflux temperature (100° C.). After 16 hrs, the reaction mixture was cooled to ambient temperature. The reaction mixture was then decanted into a 5 L fishbowl, and acetone (1950 mL) added. It was stirred for 20 min, then the orange solid was isolated by filtration and washed with acetone (3×300 mL). The resulting solid was charged to a 5 L fishbowl and H₂O (2.5 L) added. An overhead stirrer was inserted, then a solution of KPF₆ (164.76 g, 895.15 mmol) in H₂O (650 mL) was added and the orange slurry stirred for 1 hour. Then the solid was isolated by filtration and washed with H₂O (2×750 mL). The bright orange product was dried in vacuo and weighed (328.89 g, 94%).

¹H NMR (dmso-d₆, 298 K): δ 7.54 (t, 6H); 7.73 (d, 6H); 8.18 (t, 6H); 8.84 (d, 6H) ppm. ³¹P{¹H} NMR (dmso-d₆, 298 K): δ−144.21 (sept, ¹J_{PF}=710 Hz) ppm. ¹⁹F{¹H} NMR (dmso-d₆, 298 K): δ 72.51 (d, ¹J_{FP}=710 Hz) ppm. Anal. calc'd for C₃₀H₂₄F₁₂N₆P₂Ru: C 41.92%; H 2.81%; N 9.78%; P 7.21%; found: C 41.94%; H 2.75%; N 9.71%; P 7.29%

### Example 12: Representative Procedure for Intermediate Scale-Up of Synthesis of [Ru(Bpy)₃][Pf₆]₂

A 30 L jacketed reactor equipped with an overhead agitator, thermocouple, condenser and nitrogen inlet adapter was charged with [Ru(Cl)₂(p-cymene)]₂ (1155 g, 1.88 mol), 2,2′-bipyridine (1760 g, 11.25 mol) and water (7 L). The vessel was evacuated for 2 minutes with agitation then backfilled with nitrogen. This process was repeated three times. The reactor jacket was set to 115° C. and the condenser jacket was set to 20° C. The reaction mixture was heated to an internal temperature of 101° C. and stirred at this temperature for 5 h to give a dark red homogeneous solution. The reactor jacket was set to 25° C., and the mixture was cooled to below 30° C., resulting in a bright red heterogeneous slurry. A 22 L multi-necked round bottom flask equipped with an overhead agitator was charged with KPF₆ (1553 g, 8.44 mol) and water (15 L). The flask was evacuated for 2 minutes with agitation then backfilled with nitrogen. This process was repeated three times. The mixture was stirred at ambient temperature until the KPF₆ was dissolved (ca. 15 minutes). The KPF₆ solution was added to the reaction mixture over 30 minutes via peristaltic pump, and the resulting bright orange slurry was stirred at ambient temperature for 18 hrs. The slurry was filtered in a 15 L filter box, and the resulting orange solids were washed sequentially with water (2×4 L) and MTBE (7 L). The solids were dried at ambient temperature on the filter box for 16 hrs. under a sweep of nitrogen, then transferred to a vacuum oven at 45° C. for 48 hours to give the title compound as a bright orange solid (3.25 kg, 99% yield). Characterisation data are consistent with those reported in the literature. ¹H NMR (acetone d-6, 400 MHz): δ ppm 8.81 (d, 6H), 8.20 (t, 6H), 8.04 (d, 6H), 7.57 (m, 6H); ³¹P NMR (acetone d-6, 160 MHz): δ ppm −142.01 (sept, J 700 Hz); Anal. Calcd for C₃₀H₂₄F₁₂N₆P₂Ru: C, 41.92; H, 2.81; N, 9.78. Found: C, 41.76; H, 2.94; N, 9.96.

TABLE 3
Preparation of [Ru(bpy)₃][PF₆]₂ in H₂O

| | | Temp | Yield |
|---|---|---|---|
| | PF₆ | (° C.) | [Ru(bpy)₃][PF₆]₂ |
| Example | salt | of step (i) | (%) |
| 8 ^{[a]} | NH₄PF₆ | 100 | 97 |
| 9 | NH₄PF₆ | 100 | 85 |
| 10 ^{[a]} | KPF₆ | 100 | 98 |
| 11 | KPF₆ | 100 | 94 |
| 12 ^{[a]} | KPF₆ | 100 | 99 |

^{[a]} without isolation of [Ru(bpy)₃][Cl]₂•6H₂O

### Example 13: Preparation of [Ru(Phen)₃][PF₆]₂ without Isolation of Intermediate [Ru(Phen)₃][Cl]₂.6H₂O

A 20 mL scintillation vial equipped with a Teflon coated stir was charged with [Ru(p-cymene)Cl₂]₂ (612 mg, 1.0 mmol), 1,10-phenanthroline (1.08 g, 6.0 mmol), water (5.5 mL) and iPrOH (0.5 mL). The vial was sealed with a screwcap septum and evacuated with stirring until a soft boil was achieved, backfilling with N₂. This process was repeated three times. The vial was placed in an aluminum vial block pre-heated to 100° C. and stirred at this temperature for 16 h. The reaction mixture was cooled to ambient temperature, transferred to a 100 mL round bottom flask in air and diluted with water (25 mL). A separate 20 mL scintillation vial equipped with a Teflon coated stir bar was charged with KPF₆ (370 mg, 4.0 mmol) and water (5 mL). The mixture was stirred until all the KPF₆ had dissolved (ca. 5 minutes). The KPF₆ solution was transferred to the reaction mixture dropwise via syringe over 10 minutes. The resulting slurry was stirred at ambient temperature for 30 minutes. The solids were filtered over a sintered glass funnel, washed with water (3×10 mL) and dried in a vacuum over at 40° C. for 16 hours to give the product as an orange solid (1.60 g, 86%).

¹H NMR (DMSO-d₆) 400 MHz: δ ppm 8.78 (d, 6H, J=8.2 Hz), δ 8.39 (s, 6H), δ 8.09 (d, 6H J=5.2 Hz), δ 7.76 (dd, 6H, J=5.2 Hz, 8.2 Hz). ³¹P NMR (DMSO-d₆) 160 MHz: δ ppm −143.3 (sept, J_{P-F}=711 Hz). ¹⁹F NMR (DMSO-d₆) 375 MHz: δ ppm −70.13 (d, J_{P-F}=711 Hz).

### Example 14: Preparation of [Ru(Dmbpy)₃][PF₆]₂ without Isolation of Intermediate [Ru(Dmbpy)₃][Cl]₂.6H₂O

A 20 mL scintillation vial equipped with a Teflon coated stir was charged with [Ru(p-cymene)Cl₂]₂ (612 mg, 1.0 mmol), 4,4′-dimethyl-2,2′-dipyridyl (1.11 g 6.0 mmol), water (5.5 mL) and iPrOH (0.5 mL). The vial was sealed with a screwcap septum and evacuated with stirring until a soft boil was achieved, backfilling with N₂. This process was repeated three times. The vial was placed in an aluminum vial block pre-heated to 100° C. and stirred at this temperature for 16 h. The reaction mixture was cooled to ambient temperature, transferred to a 100 mL round bottom flask in air and diluted with water (25 mL). A separate 20 mL scintillation vial equipped with a Teflon coated stir bar was charged with KPF₆ (370 mg, 4.0 mmol) and water (5 mL). The mixture was stirred until all the KPF₆ had dissolved (ca. 5 minutes). The KPF₆ solution was transferred to the reaction mixture dropwise via syringe over 10 minutes. The resulting slurry was stirred at ambient temperature for 30 minutes. The solids were filtered over a sintered glass funnel, washed with water (3×10 mL) and dried in a vacuum over at 40° C. for 16 hours to give the product as an orange-red solid (1.71 g, 90%).

¹H NMR (DMSO-d6): δ ppm 8.68 (s, 6H), δ 7.54 (d, 6H, J=5.8 Hz), δ 7.33 (d, 6H, J=5.7 Hz), δ 2.51 (s, 18H). ³¹P NMR (DMSO-d₆) 160 MHz: δ ppm −143.3 (m, J_{P-F}=711 Hz). ¹⁹F NMR (DMSO-d₆) 375 MHz: δ ppm −70.10 (d, J_{P-F}=711 Hz).

### Example 15: Preparation of [Ru(Bpm)₃][PF₆]₂ without Isolation of Intermediate [Ru(Bpm)₃][Cl]₂.6H₂O

An 8 mL scintillation vial equipped with a Teflon coated stir was charged with [Ru(p-cymene)Cl₂]₂ (150 mg, 0.25 mmol), 2,2′-bipyrimidine (240 mg, 1.5 mmol), and water (2.5 mL). The vial was sealed with a screwcap septum and evacuated with stirring until a soft boil was achieved, backfilling with N₂. This process was repeated three times. The vial was placed in an aluminum vial block pre-heated to 100° C. and stirred at this temperature for 16 h. The reaction mixture was cooled to ambient temperature. A separate 8 mL scintillation vial equipped with a Teflon coated stir bar was charged with KPF₆ (185 mg, 1.0 mmol) and water (2.5 mL). The mixture was stirred until all the KPF₆ had dissolved (ca. 5 minutes). The KPF₆ solution was transferred to the reaction mixture dropwise via syringe over 5 minutes. The resulting slurry was stirred at ambient temperature for 30 minutes. The solids were filtered over a glass sintered frit and washed sequentially with water (10 mL) and methanol (2×10 mL). The resulting solids were dried in a vacuum over at 40° C. for 16 hours to give the product as a pale orange solid (320 mg, 74%).

¹H NMR (DMSO-d6): δ ppm 9.20 (d, 6H, J=3.1 Hz), δ 8.34 (d, 6H, J=4.9 Hz), δ 7.71 (t, 6H, J=5.2 Hz). ³¹P NMR (DMSO-d₆) 160 MHz: δ ppm −143.3 (m, J_{P-F}=711 Hz). ¹⁹F NMR (DMSO-d₆) 375 MHz: δ ppm −70.12 (d, J_{P-F}=711 Hz).

1.-19. (canceled)

20. A process for the preparation of a complex of formula (I): 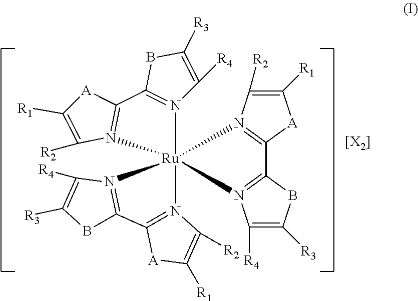
wherein R₁, R₂, R₃ and R₄ are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;
A is selected from the group consisting of:—CRₐR_{b}—, —NRₐ—, O, S, —CRₐ═CR_{b}—, —CRₐ═N—;
B is selected from the group consisting of:—CR_{c}R_{d}—, —NR_{c}—, O, S, —CR_{c}═CRₐ—, —CR_{c}═N—;
Rₐ, R_{b}, R_{c}, and R_{d} are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl, unsubstituted C₁₋₂₀-alkoxy, substituted C₁₋₂₀-alkyoxy, unsubstituted C₁₋₂₀-dialkyl amino, substituted C₁₋₂₀-dialkyl amino, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;
or Rₐ and one of R_{c} and R_{d} or R_{b} and one of R_{c} and R_{d} together with the atoms to which they are bound, form a ring; and
X is a halide;
the process comprising the step of reacting a complex of formula (II) or a complex of formula RuX₃.H₂O (IV) 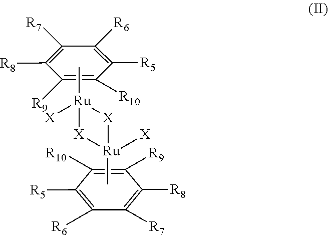
wherein R₅, R₆, R₇, R₈, R₉ and R₁₀ are independently selected from the group consisting of H, halide, unsubstituted branched or straight chain C₁₋₂₀-alkyl, substituted branched or straight chain C₁₋₂₀-alkyl, unsubstituted C₃₋₂₀-cycloalkyl, substituted C₃₋₂₀-cycloalkyl, unsubstituted C₆₋₂₀-aryl, substituted C₆₋₂₀-aryl; X is as hereinbefore defined;
with a bidentate ligand of formula (III) 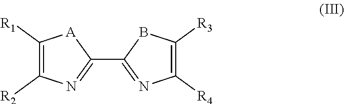
where R₁, R₂, R₃ and R₄, A and B are as hereinbefore defined;
wherein the molar ratio of the complex of formula (II): the bidentate ligand of formula (III) is about 1:6 to about 1:8 or the molar ratio of the complex of formula (IV): the bidentate ligand of formula (III) is about 1:3 to about 1:4;
wherein the process is carried out in water or a water-based solvent, wherein the water-based solvent comprises at least 60% water (by volume) and an organic solvent, at one or more temperatures in the range of about 80° C. to 110° C.

21. The process according to claim 20, wherein R₁, R₂, R₃ and R₄ are independently selected from the group consisting of H, unsubstituted branched or straight chain C₁₋₂₀-alkyl, substituted branched or straight chain C₁₋₂₀-alkyl, unsubstituted C₆₋₂₀-aryl or substituted C₆₋₂₀-aryl.

22. The process according to claim 20, wherein R₁, R₂, R₃ and R₄ are the same.

23. The process according to claim 22, wherein each of R₁, R₂, R₃ and R₄ are H.

24. The process according to claim 20, wherein A is —CRₐ═CR_{b}— or —CRₐ═N—.

25. The process according to claim 20, wherein B is —CR_{c}═CRₐ— or —CR_{c}═N—.

26. The process according to claim 20, wherein A is —CRₐ═CR_{b}— and B is —CR_{c}═CR_{d}—.

27. A process according to claim 26 wherein the ligand of formula (III) is selected from the group consisting of:
2,2′-Bipyridine;
4,4′-Bis(methyl)-2,2′-bipyridine;
4,4′-Bis(tert-butyl)-2,2′-bipyridine;
4,4′-Bis(trifluoromethyl)-2,2′-bipyridine; and
5,5′-Bis(trifluoromethyl)-2,2′-bipyridine.

28. The process according to claim 26, wherein Rₐ and one of R_{c} and R_{d} or R_{b} and one of R_{c} and R_{d} together with the atoms to which they are bound form a ring.

29. The process according to claim 28 wherein the ring is a 6-membered ring.

30. The process according to claim 29, wherein the ligand of formula (III) is:
1,10-phenanthroline;
4,7-dimethoxy-1,10-phenanthroline.

31. The process according to claim 20, wherein A is —CRₐ═N— and B is —CR_{c}═N—.

32. The process according to claim 31, wherein the ligand of formula (III) is:
2,2′-bipyrazine.

33. The process according to claim 31, wherein the ligand of formula (III) is:
2,2′-bipyrimidine.

34. The process according to claim 20, wherein X is chloride.

35. The process according to claim 20, wherein the process comprises reacting a complex of formula (II) with a bidentate ligand of formula (III) wherein the molar ratio of the complex of formula (II): the bidentate ligand of formula (III) is about 1:6 to about 1:8.

36. A process for the preparation of a compound of formula (V), 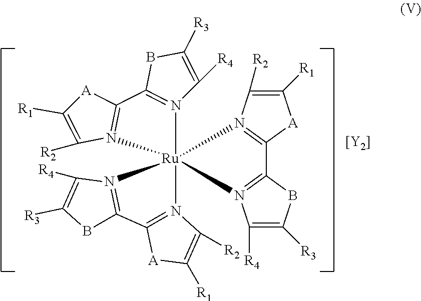
wherein R₁, R₂, R₃, R₄, A and B are as hereinbefore defined; and
Y is a non-coordinating anion or a halide which is different to X as defined in relation to the complex of formula (I);
said process comprising reacting a complex of formula (I) as hereinbefore defined with a compound of formula RY, wherein R is selected from a group consisting of an alkali metal cation, Ag⁺ and a quaternary ammonium cation and Y is as hereinbefore defined, in a molar ratio of complex of formula (I): RY of at least 1:2 and at most 1:3, wherein the process is carried out in water or a water-based solvent, wherein the water-based solvent comprises at least 60% water (volume) and an organic solvent, at one or more temperatures in the range of about 10° C. to 50° C.

37. The process according to claim 36, wherein R is K⁺, Na⁺, Ag⁺ or [R′₄N]⁺, wherein R′ is H or alkyl.

38. The process according to claim 36, wherein Y is PF₆⁻, BF₄⁻, BPh₄⁻, SbF₆⁻, [{3,5-(CF₃)₂C₆H₃}₄B]⁻, CF₃SO₃⁻, ArFSO₃⁻, [(CF₃SO₂)₂N]⁻, F, Cl, Br or I.
